(19) United States
(12) Patent Application Publication
(10) Pub. No.: US 2025/0114627 A1
(43) Pub. Date: Apr. 10, 2025

(54) TEMPERATURE CONTROL FOR MAGNETIC FLUID HYPERTHERMIA

(71) Applicants: KING FAHD UNIVERSITY OF PETROLEUM AND MINERALS, Dhahran (SA); KING ABDULLAH CITY FOR ATOMIC & RENEWABLE ENERGY (K.A. CARE), Riyadh (SA)

(72) Inventors: Sikandar KHAN, Dhahran (SA); Adeel BASHIR, Dhahran (SA); Salem BASHMAL, Dhahran (SA); Naveed IQBAL, Dhahran (SA); Sami ULLAH, Dhahran (SA)

(73) Assignees: KING FAHD UNIVERSITY OF PETROLEUM AND MINERALS, Dhahran (SA); KING ABDULLAH CITY FOR ATOMIC & RENEWABLE ENERGY (K.A. CARE), Riyadh (SA)

(21) Appl. No.: 18/752,032

(22) Filed: Jun. 24, 2024

Related U.S. Application Data
(60) Provisional application No. 63/588,868, filed on Oct. 9, 2023.

(51) Int. Cl. A61N 2/00; A61N 2/02

(52) U.S. Cl. CPC A61N 2/004; A61N 2/02

(57) ABSTRACT

A method for temperature control for magnetic fluid hyperthermia includes delivering therapeutic nanoparticles to a tumor site in a patient, exciting the therapeutic nanoparticles with a magnetic field, and regulating the temperature of the tumor with a controller. The controller is configured to constantly measure the temperature of the tumor site and increase or decrease the strength of the magnetic field to maintain the temperature of the tumor site within a therapeutic temperature range for a therapeutic time period. Further, the controller utilizes a sliding mode nonlinear control technique to regulate temperature. The sliding mode nonlinear control technique is derived from a state space representation of a system.

### CROSS REFERENCE TO RELATED APPLICATIONS

This application claims priority to U.S. Application Ser. No. 63/588,868, filed Oct. 9, 2023, the entire contents of which are hereby incorporated by reference.

### STATEMENT OF PRIOR DISCLOSURE BY AN INVENTOR

Aspects of this technology are described in an article “Designing Highly Efficient Temperature Controller for Nanoparticles Hyperthermia”, published in Nanomaterials 2022, 12, 3539, which is also incorporated herein by reference in its entirety.

### BACKGROUND

### Technical Field

The present disclosure is directed towards temperature controllers in nanotechnology, and more particularly, to a method for temperature control for magnetic fluid hyperthermia.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Cancer is one of the most common and deadliest diseases, with a high mortality rate. Different types of treatments are employed to cure this disease, such as surgery in combination with chemotherapy, immunotherapy, targeted therapy, radiation therapy, and hormone therapy. All the mentioned treatments have limitations, such as poor accessibility to tumor tissue. For example, some tumors are inoperable due to their localization [See: Dahele, M.; Brade, A.; Pearson, S.; Bezjak, A. Stereotactic radiation therapy for inoperable, early-stage non-small-cell lung cancer]. Further, the techniques mentioned above have a high degree of toxicity and are ineffective in many cases. Hyperthermia has been utilized in combination with radiotherapy as a cancer treatment for many years but poses the severe side effect of damaging healthy tissue. To overcome these limitations, scientists are constantly looking for alternative anti-tumor therapies [See: Schildkopf, P.; Ott, O. J.; Frey, B.; Wadepohl, M.; Sauer, R.; Fietkau, R.; Gaipl, U.S. Biological rationales and clinical applications of temperature-controlled hyperthermia implications for multimodal cancer treatments]. The use of nanoparticles in the field of medical science is one emerging trend. Magnetic fluid hyperthermia provides an efficient and effective solution to the issue of damaging healthy tissue (See: Ali, D.; Seyedhamidreza, E.; Milad, S.; Hossein, B.; Mohammad, H.; Reza, E.; Ali, M. A numerical investigation into the magnetic nanoparticles hyperthermia cancer treatment injection strategies), as this technique uses magnetic nanoparticles (mNPs) to remotely induce localized heating when an alternating magnetic field is applied. This may increase the temperature of the tissue harboring the tumor, as shown in FIG. 1A mNPs are injected into the tumor of a mouse which is then placed inside an alternating magnetic field (AMF).

Magnetic nanoparticles have attracted the attention of researchers worldwide due to the diversity of their applications. Most of the experiments performed with mNPs for the treatment of cancer involve direct injection of the particles into tumor tissue [See: Matsumi, Y.; Kagawa, T.; Yano, S.; Tazawa, H.; Shigeyasu, K.; Takeda, S.; Ohara, T.; Aono, H.; Hoffman, R. M.; Fujiwara, T.; et al. Hyperthermia generated by magnetic nanoparticles for effective treatment of disseminated peritoneal cancer in an orthotopic nude-mouse model]. One of the most significant factors determining the effectiveness of the method is the ability of the mNPs to be easily driven and collected in the target organ. Magnetic hyperthermia (MHT) is a promising approach for cancer therapy, for example maghemite (γ-Fe₂O₃) nanoparticles have been used for hyperthermia in the treatment of cancer. The nanoparticles used for magnetic fluid hyperthermia should respond to physical-chemical properties such as aggregation state, heat dose production, heat conversion from magnetic energy, and surface chemistry. Similarly, superparamagnetic iron oxide nanoparticles (SPIONs) are also a good option due to their biocompatibility, excellent response to external magnetic field, non-toxic nature, and ease of production [See: Rezanezhad, A.; Hajalilou, A.; Eslami, F.; Parvini, E.; Abouzari-Lotf, E.; Aslibeiki, B. Superparamagnetic magnetite nanoparticles for cancer cells treatment via magnetic hyperthermia: Effect of natural capping agent, particle size and concentration. J. Mater. Sci. Mater. Electron].

The process of killing tumor cells through hyperthermia is a two-step process. In the first step, cells undergo an increased metabolic rate, which in turn increases the generation of reactive oxygen species; the second step then causes protein damage through oxidation, aggregation, and denaturation. Hyperthermia leads to nuclear protein damage which then inhibits DNA repair mechanisms. Similarly, hyperthermia causes cell proliferation via G1 cell cycle arrest or mitotic catastrophe. Further, hyperthermia may also lead to membrane damage, which alters the transport function, signaling mechanism, and/or receptor function of the cell. All of the aforementioned cell changes may lead to the death of the tumor cell (FIG. 1B).

In tissue, heat is generated by metabolism and blood perfusion. The heat generated during metabolic processes, such as growth and energy production of the living system, is defined as metabolic heat. The effects of temperature increase during the metabolism of material can be reduced by lowering the frequency of the alternating magnetic field. Hence, the net heat will remain the same. There is a need for a simple analytical solution to evaluate the effect of parameters such as metabolic heat generation during hyperthermia. One promising feature of such a technique is that it may be used in regions that are difficult to access owing to the intravenous administration route of mNPs [See: Chang, D.; Lim, M.; Goos, J.; Qiao, R.; Ng, Y. Y.; Mansfeld, F. M.; Jackson, M.; Davis, T. P.; Kavallaris, M. Biologically Targeted Magnetic Hyperthermia: Potential and Limitations].

In the event the human body is heated past 43 degrees Celsius (° C.), serious interruptions may occur in life-support systems, and heat stroke symptoms develop. Further increases in temperature may cause irreversible damage of the structure and function of protein molecules, leading to tissue death. In this regard, attention is drawn towards killing cancerous tissues using local hyperthermia. Many researchers have defined the anti-cancerous activity that occurs between 41° C. and 43° C. as minor hyperthermia, and greater than or equal to 50° C. as thermal ablation treatment. However, there are only a few explorations which include a direct comparison of the heating doses' effectiveness during magnetic hyperthermia, 43° C. with 50° C. and similarly, 50° C. with 60° C. to 70° C. Accordingly, temperature must be controlled precisely in local hyperthermia so only tumor tissue is killed, and healthy tissue is unaffected.

Magnetic fluid hyperthermia (MFH) has shown positive results in recent clinical experiments. To restrict heat diffusion, accurate measurement of the temperature and control of the magnetization current is important. The principle of this phenomenon is the fact that tumor cells are more sensitive to temperature changes than local cells. The local cells are those that are healthy or free from any sort of mutation. Further, clinical studies have proven that the walls of mutated cells are more porous than normal cells, as shown in FIG. 1C, allowing easy deposition of nanoparticles on tumor cells. Once the nanoparticles reach the desired tissue, they can be placed under a varying magnetic field to dissipate the heat locally, and the temperature is raised to 45° C. to kill cancerous cells. The set point of the temperature controller is 45° C. and is completely safe for normal tissue. One of the challenging tasks of the method is maintaining the temperature strictly at the desired point i.e., 45° C. A temperature controller for magnetic fluid hyperthermia provides the precise temperature control needed to avoid the folding of proteins and prevent tissue destruction around cancerous tissue.

Although several methods/hyperthermic devices to control temperature at the tumor site have been developed to treat cancer in the past, each of these methods suffers from certain drawbacks inhibiting their adoption. Accordingly, an object of the present disclosure is to provide a method and a system that allows for precise control of temperature in magnetic fluid hyperthermia in applications where it is necessary to heat a specific area in a controlled way, thereby circumventing the drawbacks of prior art.

### SUMMARY

In an exemplary embodiment, a method for temperature control for magnetic fluid hyperthermia is disclosed. The method includes injecting therapeutic nanoparticles into a tumor site in a patient, exciting the therapeutic nanoparticles with a magnetic field, and regulating the temperature of the tumor with a controller. The controller is configured to constantly measure the temperature of the tumor site and increase or decrease the strength of the magnetic field to maintain the temperature of the tumor site within a therapeutic temperature range for a therapeutic time period. Further, the controller utilizes a sliding mode nonlinear control technique to regulate temperature. The sliding mode nonlinear control technique is derived from a state space representation of a system including the therapeutic nanoparticles, the magnetic field, and the controller.

In some embodiments, the therapeutic temperature range is between 44.0 degrees Celsius (° C.) and 46.0° C. In some embodiments, the therapeutic nanoparticles are excited to 45° C.

In some embodiments, the temperature that the therapeutic nanoparticles are excited to, is a temperature within the therapeutic temperature range.

In some embodiments, the controller includes a magnetic coil, a power source, a temperature probe, and processing circuitry.

In some embodiments, the controller utilizes state space calculations to regulate temperature.

In some embodiments, the steady state error is zero.

In some embodiments, the therapeutic nanoparticles comprise of magnetic nanoparticles.

In some embodiments, the magnetic nanoparticles comprise of superparamagnetic iron oxide. In some embodiments, the magnetic nanoparticles are comprised of ferrite.

In some embodiments, heat produced by excited therapeutic nanoparticles kills cancerous cells without killing non-cancerous cells.

In another exemplary embodiment, an apparatus for regulating the temperature of therapeutic nanoparticles disposed within a tumor is disclosed. The apparatus includes a power source configured to supply an electric current, a magnetic coil configured to convert the electric current into a magnetic field, and a controller configured to regulate the strength of the magnetic field. The apparatus further includes a temperature probe configured to indirectly measure the temperature of the therapeutic nanoparticles by measuring the temperature of the tumor. The magnetic field may excite therapeutic nanoparticles to a target therapeutic temperature and the controller is further configured to maintain the temperature of the therapeutic nanoparticles at the target therapeutic temperature.

In some embodiments, the target therapeutic temperature is 45° C.

In some embodiments, the controller utilizes a non-linear control technique.

In some embodiments, the nonlinear control technique is sliding mode control.

In some embodiments, the sliding mode control is derived from a state space representation of a system, the system including the apparatus and therapeutic nanoparticles disposed within the tissue of a patient.

In some embodiments, the steady-state error is zero.

In yet another exemplary embodiment, a method of temperature control for magnetic fluid hyperthermia is disclosed. The method includes injecting therapeutic nanoparticles to a tumor site in a patient and exciting the therapeutic nanoparticles with a magnetic field. Further, the method includes regulating the temperature of the tumor site with a controller, the controller being configured to constantly measure the temperature of the site. The controller is configured to increase or decrease the strength of the magnetic field to maintain the temperature of the tumor site at 45° C. The controller utilizes a sliding mode control derived from a state space representation of a system. The aforementioned system includes the therapeutic nanoparticles, the site of the tumor, the magnetic field, and the controller; and the therapeutic nanoparticles are comprised of superparamagnetic iron oxide.

In some embodiments, the steady state error is zero.

In some embodiments, heat produced by excited therapeutic nanoparticles kills cancerous cells without killing noncancerous cells.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic diagram depicting tumor heating in a magnetic field, according to certain embodiments.

FIG. 1B is a flowchart depicting induced cell changes due to hyperthermia, according to certain embodiments.

FIG. 1C is a schematic diagram depicting accumulations of nanoparticles in tumor, according to certain embodiments.

FIG. 2 is a method flowchart for temperature control for magnetic fluid hyperthermia, according to certain embodiments.

FIG. 3 depicts a biocompatible shell for nanoparticles, according to certain embodiments.

FIG. 4 is a block diagram depicting a controller system for magnetic fluid hyperthermia, according to certain embodiments.

FIG. 5 is a block diagram depicting a Simulink model of a plant with a proportional- integral-derivative (PID) controller, according to certain embodiments.

FIG. 6A depicts a step response of the plant using a proportional-integral (PI) controller, according to certain embodiments.

FIG. 6B depicts a step response of the plant using the PID controller, according to certain embodiments.

FIG. 6C depicts a step response of the plant using a proportional-derivative (PD) controller, according to certain embodiments.

FIG. 7 depicts a step response of a system with the PI controller, according to certain embodiments.

FIG. 8 is a block diagram depicting the Simulink model with a pole placement controller, according to certain embodiments.

FIG. 9 depicts a step response of the pole placement controller, according to certain embodiments.

FIG. 10 is a block diagram depicting the Simulink model of a plant with a sliding mode controller (SMC), according to certain embodiments.

FIG. 11 depicts a step response of the plant with the SMC controller, according to certain embodiments.

FIG. 12 depicts a comparison in the step response of the plant with the SMC controller, the pole placement controller, and the PD controller, according to certain embodiments.

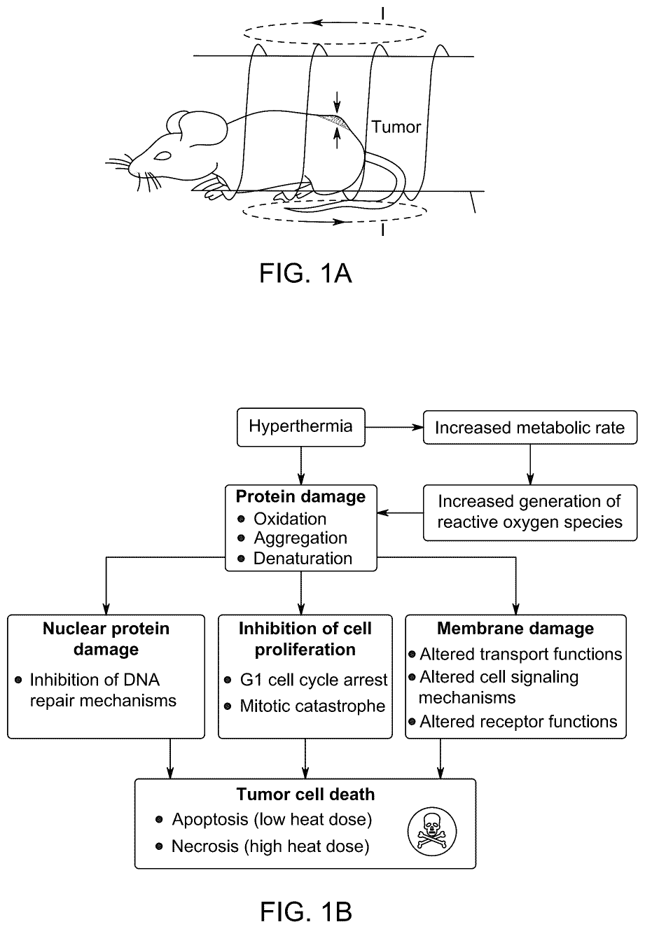

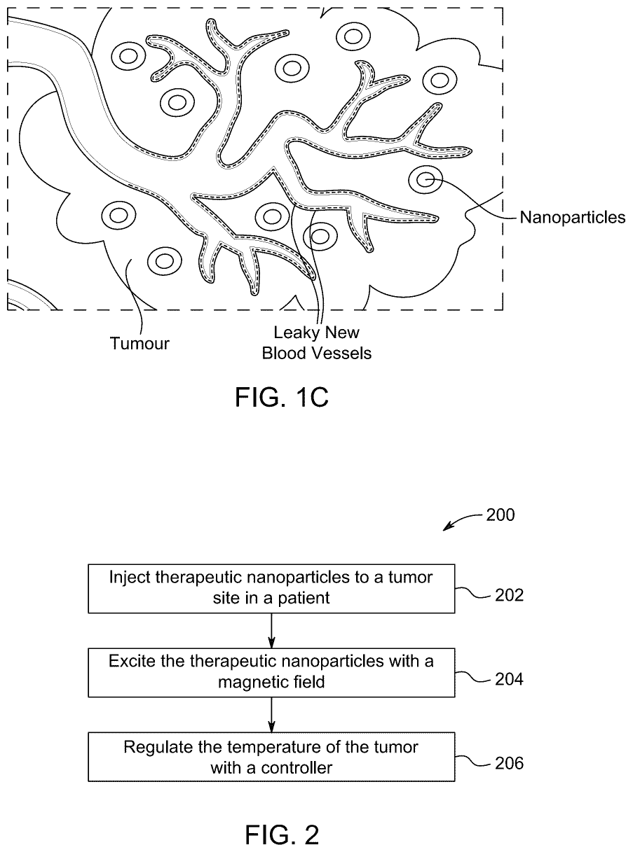

### DETAILED DESCRIPTION

When describing the present disclosure, the terms used are to be construed in accordance with the following definitions, unless context dictates otherwise.

Embodiments of the present invention will now be described more fully hereinafter with reference to the accompanying drawings wherever applicable, in that some, but not all embodiments of the disclosure are shown.

In the drawings, like reference numerals designate identical or corresponding parts throughout the several views. Further, as used herein, the words “a,” “an” and the like generally carry a meaning of “one or more,” unless stated otherwise.

Furthermore, the terms “approximately”, “approximate”, “about,” and similar terms generally refer to ranges that include the identified value within a margin of 20%, 10%, or preferably 5%, and any values therebetween.

The use of the terms “include,” “includes”, “including,” “have,” “has,” or “having” should be generally understood as open-ended and non-limiting unless specifically stated otherwise.

As used herein, “nanoparticles” are particles having a particle size of 1 nm to 500 nm within the scope of the present invention. The nanoparticles may exist in various morphological shapes, such as nanowires, nanosheets, nanocrystals, nanorectangles, nanotriangles, nanopentagons, nanohexagons, nanoprisms, nanodisks, nanocubes, nanoribbons, nanoblocks, nanobeads, nanotoroids, nanodiscs, nanobarrels, nanogranules, nanowhiskers, nanoflakes, nanofoils, nanopowders, nanoboxes, nanostars, tetrapods, nanobelts, nano-urchins, nanoflowers, etc., and mixtures thereof.

As used herein, “magnetic materials” refers to materials that are impacted by external electromagnetic fields in their surroundings.

As used herein, “magnetic nanoparticles” refers to a class of nanoparticles that can be manipulated using magnetic fields. They consist of a magnetic material, such as iron, nickel, and cobalt, and a chemical component with functional groups.

As used herein, “magnetic fluid hyperthermia” refers to the conversion of heat from magnetic nanoparticles via magnetic energy loss in the presence of an external applied magnetic field.

Aspects of the present disclosure are directed to a system and method that includes using a cost-effective, efficient, and practically implementable temperature controller for magnetic fluid hyperthermia (MFH). The principle of effective implementation of the apparatus and method centers on the greater sensitivity of tumor cells to changes in temperature, in comparison to healthy cells. Once the nanoparticles reach the desired tissue, they are exposed to a varying magnetic field to deliver heat locally thereby raising the temperature of the target tissue to about 45° C. to kill only cancerous cells. One challenging task is maintaining the temperature strictly at the desired point, i.e., 45° C. Conventionally, linear control strategies have been adopted to control temperature to avoid the folding of proteins and save healthy tissue around cancerous tissue from getting destroyed; however, these methods suffer from certain drawbacks. The present disclosure relies on a non-linear control technique like sliding mode control (SMC) to maintain the temperature accurately at the desired value. A comparison herein of a non-linear control technique with conventional control techniques shows that the method and system of the present disclosure provide substantial improvements in settling time and rise time. Steady-state error was also reduced to zero using this technique. The method of the present disclosure may be used as a substitute for conventional radiation and chemotherapy treatment of cancer.

FIG. 2 illustrates a flow chart of a method 200 for temperature control for magnetic fluid hyperthermia. The order in which the method 200 is described is not intended to be construed as a limitation, and any number of the described method steps can be combined in any order to implement the method 200. Additionally, individual steps may be removed or skipped from the method 200 without departing from the spirit and scope of the present disclosure.

At step 202, the method 200 includes delivering therapeutic nanoparticles to a tumor site in a patient, e.g., by injection. The therapeutic nanoparticles comprise magnetic nanoparticles. Therapeutic nanoparticles refer to a new class of therapeutics for cancer which can perform in ways that other therapeutic modalities cannot. Therapeutic nanoparticles show an enhanced efficacy with lower side effects than traditional small-molecule chemotherapeutics. In some embodiments, the therapeutic nanoparticles contain magnetic nanoparticles. In some embodiments, the magnetic nanoparticles may contain a diamagnetic, paramagnetic, superparamagnetic, or ferromagnetic material that is responsive to a magnetic field. Non-limiting examples of therapeutic magnetic nanoparticles include a core of a magnetic material containing a metal oxide selected from the group of magnetite; ferrites (for e.g., ferrites of manganese, cobalt, and nickel); Fe(II) oxides, and hematite, and metal alloys thereof. In a specific embodiment, the magnetic nanoparticles include superparamagnetic iron oxide. In yet another embodiment, the magnetic nanoparticles include ferrite.

At step 204, the method 200 includes exciting the therapeutic nanoparticles with a magnetic field. In some embodiments, the temperature that the therapeutic nanoparticles are excited to is a temperature within the therapeutic temperature range. In some embodiments, the therapeutic nanoparticles are excited to a range between 44.0-46.0 degrees Celsius (° C.), preferably 44.2-45.8° C., preferably 44.4-45.6° C., preferably 44.6-45.4° C., and preferably 44.8-45.2° C. In a specific embodiment, the therapeutic nanoparticles are excited to 45° C. In some embodiments, heat produced by excited therapeutic nanoparticles kills cancerous cells without killing non-cancerous cells.

At step 206, the method 200 includes regulating the temperature of the tumor with a controller. The temperature of the tumor is regulated by the controller. The controller includes a magnetic coil, a power source, a temperature probe, and processing circuitry. The controller is configured to constantly measure the temperature of the tumor site and regulate the strength of the magnetic field to maintain the temperature of the tumor site within a therapeutic temperature range for a therapeutic time. For instance, the controller may be configured to increase/decrease the strength of the magnetic field to maintain the temperature of the tumor site within the therapeutic temperature range for the therapeutic time. The therapeutic temperature range is between 44.0-46.0 degrees Celsius (° C.), preferably 44.2-45.8° C., preferably 44.4-45.6° C., preferably 44.6-45.4° C., and preferably 44.8-45.2° C.

In some embodiments, the controller utilizes state space calculations to determine the required variation of the magnetic field to achieve the temperature threshold. Once the threshold temperature is reached, the controller stops the field variation so that the temperature does not exceed a pre-set value. In some embodiments, the controller utilizes a sliding mode nonlinear control technique to regulate temperature. The sliding mode nonlinear control technique is derived from a state space representation of a system. The system includes the therapeutic nanoparticles, the magnetic field, and the controller. The steady-state error by the system/method of the present disclosure is zero.

The state space representation of the system may comprise a mathematical model of the physical system, including the therapeutic nanoparticles, the magnetic field, and the controller, expressed as a function of input, output, and state variables related by first-order differential equations or difference equations. The sliding mode control technique can be represented by a derivation of the control input “U” and may have a sliding surface defined by:

$$S = e {( d / dt + λ )}^{n - 1}$$

$$e = {T}_{d} - {T}_{m}$$

Wherein, S=Sliding surface; e=error; Td=Desired temperature value; Tm=Measured temperature value; and λ=Tuning parameter. The sliding mode control technique may provide for regulation of the overall temperature of the system at the desired temperature value with minimal oscillation or variation in temperature as the measured temperature may “slide” along the sliding surface defined above during operation of the system. The regulation of temperature may occur through the manipulation of any characteristic(s) of the magnetic field including but not limited to, strength, amplitude, frequency, direction, and any variations or combinations thereof.

Excitation may comprise any changes in the magnetic, electromagnetic, and/or energy level of a particle, including but not limited to, any changes which may occur as a result of the application of a magnetic field(s).

In another aspect, an apparatus for regulating the temperature of therapeutic nanoparticles disposed within a tumor is described. The apparatus includes a power source, a magnetic coil, a controller, and a temperature probe. The power source is configured to supply an electric current, and the magnetic coil is configured to convert the electric current into a magnetic field. The magnetic field may excite therapeutic nanoparticles to a target therapeutic temperature. The therapeutic nanoparticles effectively kill cancerous cells at the target therapeutic temperature. In some embodiments, the strength of the magnetic field is periodically regulated to prevent overheating. The controller is configured to regulate the strength of the magnetic field, and the temperature probe is configured to indirectly measure the temperature of the therapeutic nanoparticles by measuring the temperature of the tumor and/or the tissue around it. Once the temperature probe determines the temperature of the tumor, the controller may increase/decrease the strength of the magnetic field based on the input received from the temperature probe. In some embodiments, the controller is further configured to maintain the temperature of the therapeutic nanoparticles at the target therapeutic temperature. In some embodiments, the therapeutic temperature range is between 44.0-46.0° C., preferably 44.2-45.8° C., preferably 44.4-45.6° C., preferably 44.6-45.4° C., and preferably 44.8-45.2°° C. In some embodiments, the target therapeutic temperature is 45° C. This is achieved by utilizing a non-linear control technique. The non-linear control technique is sliding mode control (SMC). In some embodiments, the sliding mode control is derived from a state space representation of a system, the system comprising the apparatus and therapeutic nanoparticles disposed within the tissue of a patient. In some embodiments, the steady- state error is zero.

### EXAMPLES

The following details of the examples demonstrate a method for temperature control for magnetic fluid hyperthermia as described herein. The examples are provided solely for illustration and are not to be construed as limitations of the present disclosure, as many variations thereof are possible without departing from the spirit and scope of the present disclosure.

### Example 1: Delivery of Nanoparticles to a Tumor and a Tumor Site

There are at least two ways in which nanoparticles can be delivered to sites of cancerous tissue, i.e., direct injection of nanoparticles into the cancerous tissue and intravenous injection. Direct injection of nanoparticles into the cancerous tissue is less suited for the following reasons. Firstly, it is challenging to determine whether all the particles injected have reached the affected tissue. Secondly, if the tumor lies under a sensitive organ, it might be difficult to get to the affected tissues. The injection of particles directly into tissue comes with other problems, including potentially missing an area of the tumor, thus allowing cancerous cells to re-grow after some time.

Intravenous injection is a technique that has been used for centuries for the delivery of drugs and is now used for magnetic fluid hyperthermia [See: Huang H S, Hainfeld J F. Intravenous magnetic nanoparticle cancer hyperthermia. Int J Nanomedicine. 2013, 8, 2521, doi: 10.2147/IJN.S43770, incorporated herein by reference in its entirety.]

For the purposes of this disclosure, “injecting therapeutic nanoparticles to a tumor site” may be understood to encompass both direct injection of nanoparticles and intravenous injection of nanoparticles, so long as the nanoparticles ultimately reach a tumor site.

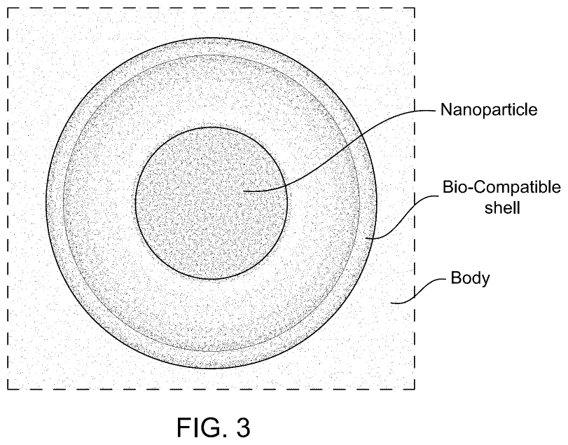

Laboratory experiments show that the accumulation of nanoparticles is 16% greater in the case of intravenous injection than in direct injection of the particles into the tumor. However, a challenge in administering these particles intravenously is the potential for iron toxicity. Scientists have sought to address this issue by wrapping the particles in a biocompatible shell to alleviate their toxicity, as shown in FIG. 3. This increases the biocompatibility of superparamagnetic core-shell nanoparticles for their future use in magnetic fluid hyperthermia. Similarly, nanoparticles may be synthesized from manganese ferrite with a biocompatible shell composed of polyvinylpyrrolidone (PVP).

As shown in FIG. 3, the particles inside the shell will do not come into direct contact with the body, therefore toxicity is avoided. Different types of polymers can be used to coat the nanoparticles; some are given in Table 1. In the present disclosure, commercially available spherical SPIONs (superparamagnetic iron oxide) from Skyspring Nanomaterials Inc., Houston, Texas, USA (3327NG.) nanoparticles are used, whose average size is 10 nm to 15 nm.

TABLE 1
Different materials used for coating of nanoparticles.

| No. | Material | Reference |
|---|---|---|
| 1 | Gelatin | [50] |
| 2 | Dextran | [51] |
| 3 | Polyvinyl alcohol | [52] |
| 4 | Polyethylene glycol | [53] |
| 5 | Chitosan | [54] |
| 6 | Polyacrylic acid | [55] |
| 7 | Polyvinylpyrrolidone | [56] |
| 8 | Poly(D, L-lactide) | [57] |

50. Pathania, D.; Kumar, S.; Thakur, P.; Chaudhary, V.; Kaushik, A.; Varma, R. S.; Furukawa, H.; Sharma, M.; Khosla, A. Essential oil-mediated biocompatible magnesium nanoparticles with enhanced antibacterial, antifungal, and photocatalytic efficacies. Sci. Rep. 2022, 12, 1-13.
51. Fatima, H.; Charinpanitkul, T.; Kim, K.-S. Fundamentals to Apply Magnetic Nanoparticles for Hyperthermia Therapy. Nano-materials 2021, 11, 1203.
52. Divya, M.; Vasecharan, B.; Abinaya, M.; Vijayakumar, S.; Govindarajan, M.; Alharbi, N. S.; Kadaikunnan, S.; Khaled, J. M.; Ben-elli, G. Biopolymer gelatin-coated zinc oxide nanoparticles showed high antibacterial, antibiofilm and anti-angiogenic activity. J. Photochem. Photobiol. B: Biol. 2018, 178, 211-218.
53. Chircov, C.; Ştefan, R.-E.; Dolete, G.; Andrei, A.; Holban, A. M.; Oprea, O.-C.; Vasile, B. S.; Neacşu, I. A.; Tihǎuan, B. Dextran-Coated Iron Oxide Nanoparticles Loaded with Curcumin for Antimicrobial Therapies. Pharmaceutics 2022, 14, 1057.
54. Ghanaatian, A.; Elhambakhsh, A.; Bakhtyari, A.; Ghasemi, M. N.; Esmaeilzadeh, F.; Vakili-Nezhaad, G. R. Coating SiO₂ nano-particles with polyvinyl alcohol for interfacial tension alteration in the system CO₂ + 17olyethylene glycol + water. Surfaces Interfaces 2022, 32.
55. Shi, L.; Zhang, J.; Zhao, M.; Tang, S.; Cheng, X.; Zhang, W.; Li, W.; Liu, X.; Peng, H.; Wang, Q. Effects of pol-yethylene glycol on the surface of nanoparticles for targeted drug delivery. Nanoscale 2021, 13, 10748-10764.
56. Frank, L. A.; Onzi, G. R.; Morawski, A. S.; Pohlmann, A. R.; Guterres, S. S.; Contri, R. V. Chitosan as a coating material for nano-particles intended for biomedical applications. React. Funct. Polym. 2020, 147, 104459.
57. Arkaban, H.; Barani, M.; Akbarizadeh, M. R.; Chauhan, N. P. S.; Jadoun, S.; Soltani, M. D.; Zarrintaj, P. Polyacrylic Acid Nano-platforms: Antimicrobial, Tissue Engineering, and Cancer Theranostic Applications. Polymers 2022, 14, 1259, each incorporated herein by reference in its entirety.

### Example 2: Feasible Temperature Measuring Techniques for Magnetic Fluid Hyperthermia

The first and most important task in applying a magnetic fluid hyperthermia technique is to find an efficient way to measure the temperature of the tumor site. Although there are many techniques to measure temperature, it is tough to find a technique that suits the requirements at the tumor site. The temperature-measuring technique preferably has at least six properties specified hereinafter. The temperature-measuring technique must measure temperature at depth, have high accuracy, be non-invasive, may be used practically with ease, must not be bulky, and must be economical.

Different types of measuring instruments and techniques are available for the measurement of temperature. For instance, miniature compact thermometers can be used for temperature measurement. They have an almost 95% accuracy, are not bulky, and are economical. However, one of the major reasons they cannot be used in magnetic fluid hyperthermia is that miniature compact thermometerss cannot measure temperature at depth and miniature compact thermometers are invasive. Similarly, ultrasound methods are also sometimes used for temperature measurement. Ultrasound methods are non-invasive, portable, not bulky, and can measure temperature at depth. However, the temperature sensitivity of ultrasound techniques are much lower than is required so they cannot be used for the desired treatment. Another temperature- measuring technique, magnetic resonance imaging (MRI), is highly accurate compared to the other methods described above. However, the major disadvantage of this technique is that it requires the use of bulky equipment. The fiber-optic temperature measurement technique has also attracted the attention of researchers; however, one major drawback of this technique is that the accuracy of sensors varies widely. Additionally, the development of the sensor is highly complex, and some are not economical. Thermal-imaging infrared cameras may be considered as potential candidate for magnetic fluid hyperthermia, but their accuracy is hindered by differing emissivity and reflections from surfaces. Moreover, it is a costly technique and requires a high initial investment.

Temperature measurement utilizing the photoacoustic effect may address some of these challenges. In this technique, the temperature is measured by using the following formula as described below as Equation 1:

$$T = \frac{P}{P ( T ° )} ( \frac{A}{B} + T ° ) - \frac{A}{B}$$

where T is equal to the measured temperature, P is equal to the amplitude of the acoustic wave, A and B are equal to constants determined by the material, P) (T°) is equal to the known reference point of temperature, and T° is equal to the initial temperature. This technique is non-invasive, economical, and practical for magnetic fluid hyperthermia.

A temperature probe may be any device, or component thereof, capable of sensing local temperature data. A temperature probe may be a miniature compact thermometer, an MRI, a fiber-optic thermometer, a laser thermometer, a thermal-imaging infrared camera, an acoustic wave temperature sensing device, and/or a photoacoustic effect sensitive temperature sending device.

### Example 3: System Modelling

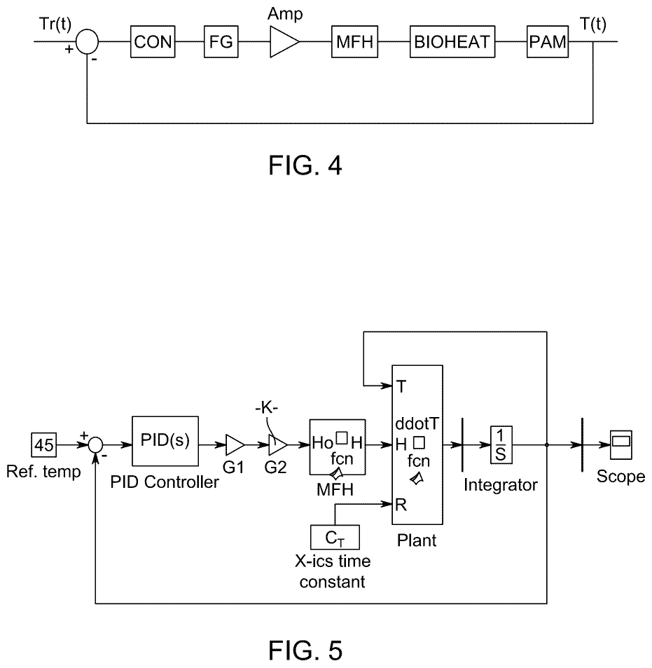

After selecting the appropriate technique for temperature measurement, the next step is to design the controller for magnetic fluid hyperthermia. The controller acts as the brain of this system. A block diagram of the system is shown in FIG. 4. As can be seen from FIG. 4, the block diagram includes a controller (CON), a function generator (FG), an amplifier (AMP), magnetic ferrofluid heating (MFH), bio heating effect (BIOHEAT), photoacoustic measurement (PAM), reference temperature (Tr(t)), and measured temperature (T(t)).

The controller is the brain of the system, as it regulates the temperature of the system. The controller determines on the magnetic field variation necessary to achieve the threshold temperature and then ceases field variation so that the temperature does not exceed the pre-set value for temperature. In this way, the controller saves adjacent tissues from destruction. The function generator used in this system obtains the power value from the control unit and produces a physical radio frequency to feed the amplifier. The amplifier used is a power amplifier with unity gain. These two can be merged into one block with power gain (Gi). MFH describes the phenomena behind the heating of the nanoparticles under a magnetic field. Ferrofluid refers to a liquid which is or may be strongly magnetized in the presence of a magnetic field. As nanoparticles are injected within a mixture of liquid, they become magnetized in the presence of the magnetic field. When placed in an alternating magnetic field, magnetic nanoparticles (mNPs) undergo Brownian and

Neel relaxation processes, resulting in energy absorption from the magnetic field; this energy is responsible for heating the ferrofluid. The volumetric power deposition is given by U in Equation 2. The heating of the fluid is governed by the first law of thermodynamics. The mathematical Equation used for the ferrofluid is given below as Equation 2:

$$U = \frac{\{ ( \frac{U}{2} ) * {H}_{0}^{2} * {X}_{0} * {ω}^{2} * {T}_{a} \}}{\{ 1 + {( ω * {T}_{a} )}^{2} \}}$$

where, U is equal to volumetric power deposition, μ is equal to permeability of free space (F/m), H₀ is equal to magnetic field strength, X₀ is equal to magnetic susceptibility, ω is equal to radian frequency, and Tₐ is equal to effective relaxation time. Further, the most complex part of the system pertains to bioheat; a mathematical model of the tissue where the tumor is located. The basic characteristics of all tissue in the context of this system are roughly the same so one generic model can be used. Changes in the system will depend on various parameters, such as, but are not limited to, nanoparticle heating, blood convection, and heat diffusion.

Considering all the parameters mentioned above, Equation 3 may be used for modeling the bioheat as described below:

$$ρ ( r ) c ( r ) [ \frac{\partial T ( r , T )}{\partial t} ] = ∇ K ( r ) * ∇ T ( r \cdot t ) + U ( r , t ) + {c}_{b} ω ( {T}_{a} - T ( r , t ) )$$

where, ρ(r) is equal to the mass density of nanoparticle-laden tissue; c(r) is equal to the specific heat of nanoparticle-laden tissue; K(r) is equal to the thermal conductivity of nanoparticle laden tissue, c_{b} is equal to the specific heat of blood, ω is equal to local perfusion rate of blood, and Tₐ is equal to temperature of the blood. Further, T(r, t) is equal to temperature inside the patient, and U(r, t) is equal to the time rate of electromagnetic energy deposited per unit volume. Furthermore, Equation 3 is simplified in order to obtain an appropriate analytical solution for the system. If there is a sharp roll-off of the temperature in the tissues laden with nanoparticles, then the Equation simplifies to Equation 4 as described below:

$$ρ c [ \frac{\partial T ( T )}{\partial t} ] = - R ( T ( t ) - {T}_{a} ) + U ( t ) + {c}_{b} ω ( {T}_{a} - T ( t ) )$$

where R is equal to the characteristic time constant of the tissue nanoparticle system, and all other symbols have the same meaning as described in Equation 3 above. With all blocks modelled and after taking their Laplace, the entire system can be represented with a transfer function given below as Equation 5:

$${Q}_{a} = [ \frac{\frac{{G}_{1} {G}_{2}}{ρ c}}{s + \frac{β}{ρ c}} ] {e}^{- sL}$$

where, β is equal to local blood perfusion constant, G1 is equal to power amplifier gain, and G2 is equal to gain for local thermal diffusion.

### Example 4: Controller Design (PID Controller)

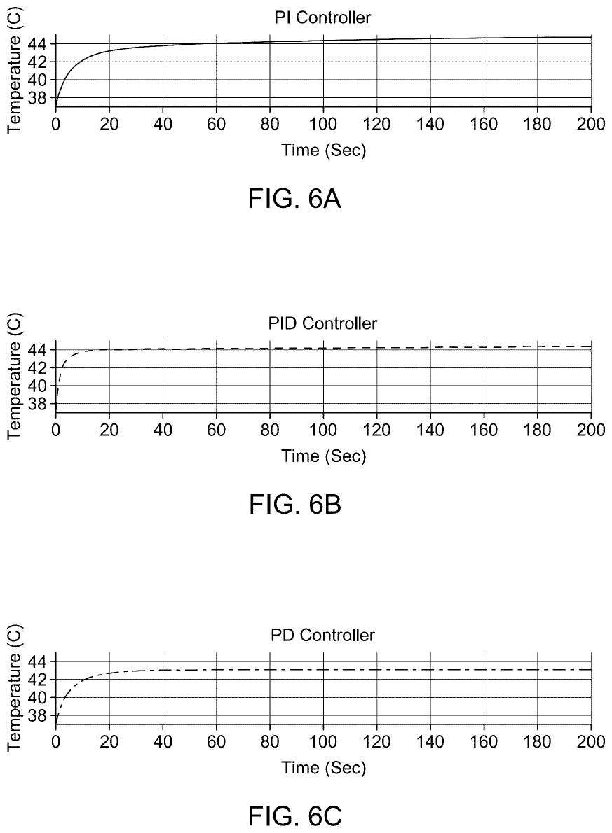

In total, three different types of control techniques are applied to the above-modeled system, their simulation and results are explained herein, and their comparison is provided. In nanoparticle hyperthermia temperature control, all three combinations of the PID controller may be applied. MATLAB Simulink (R2021a) is used to define the system transfer function as functional blocks, and then a proportional-integral-derivative (PID) controller block is linked to the input of the plant, as shown in FIG. 5. The value of the preset temperature may be set to 45° C. After adjusting the parameters and tuning the PID parameters, a step response of the system may be obtained from the scope. The step response of the plant using a proportional-integral (PI) controller, proportional-derivative (PD) controller, and PID controller is shown in FIG. 6A, FIG. 6B, and FIG. 6C, respectively. It can be seen from FIGS. 6A-6C that only the PI controller reaches the desired temperature of 45° C., whereas PID reaches 44° C. and PD reaches 43° C. So, only the PI controller is further discussed.

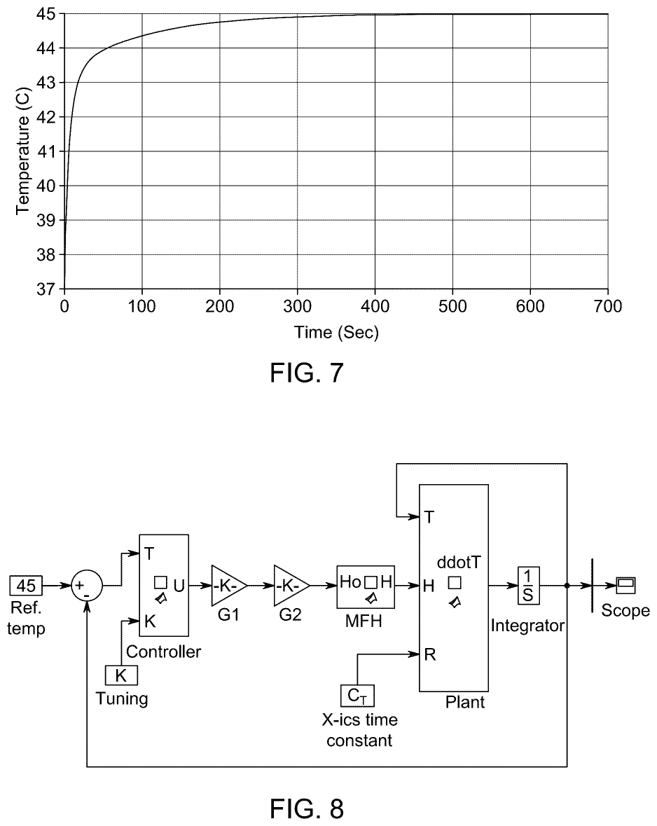

There are two parameters that are important to the functioning of the PI controller: one is the rise time of the system, and the other is the settling time. The system should reach and settle on the desired value as fast as possible with no overshoot in temperature. Overshooting of the temperature may be lethal. The PI may fine-tuned to attain the desired results. Manual tuning methods may be used to tune the parameters of the Kp and Ki to achieve the desired response. The system should reach and settle on the desired value as fast as possible with no overshoot in temperature. The response of the system is shown in FIG. 7. It may be seen from the response that the rise time of the system may preferably be between 10 and 1000 seconds, the rise time may preferably be between 50 and 500 seconds, the rise time may preferably be between 75 and 250 seconds the rise time may preferable be 100 seconds. The settling time of the system may preferably be between 100 and 1000 seconds, the settling time of the system may preferably be between 250 and 750 seconds, the settling time of the system may preferably be 500 seconds.

### Example 6: Pole Placement Technique

The idea behind the pole placement technique is to transform the state-space representation of the system into the standard form as given below,

$$x = Ax + Bu$$

$$y = Cx + Du$$

where A is equal to the state matrix, B is equal to the input matrix, C is equal to the output matrix, D is equal to the feed through the matrix, u is equal to the input, y is equal to the output, and x is equal to the state vector. Further, the roots of the system are calculated, and then full-state feedback is used to form the general Equation given below for the K matrix. This K matrix is used for controlling purposes:

$$u = - Kx$$

where, K is the feedback matrix. The Simulink model (R2021a) of the complete nanoparticles hyperthermia system with pole placement controller design is shown in FIG. 8.

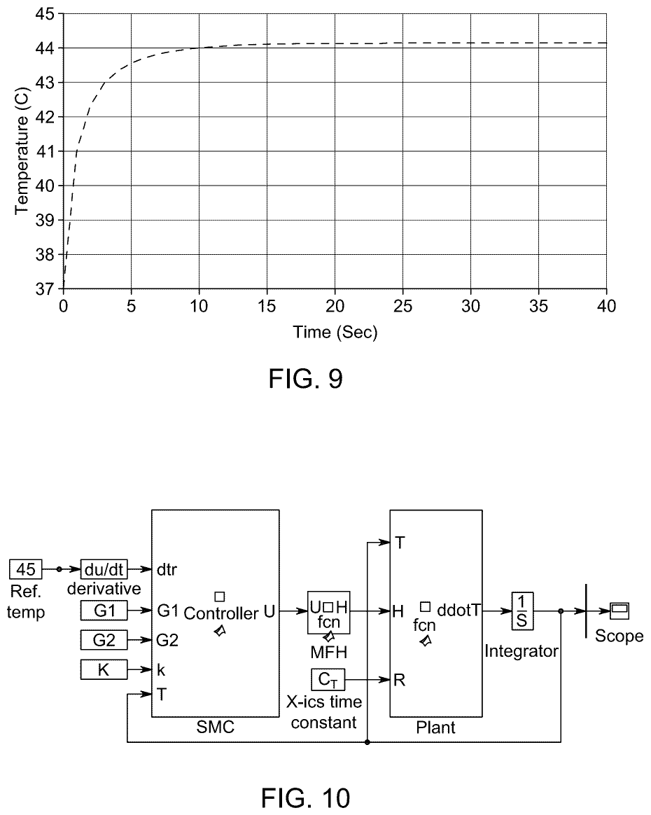

In the Simulink model, it is shown that error is calculated from a pre-set value of the temperature and from the actual temperature value measured from the target tissue. The measured error value is then fed to the pole placement controller block. Depending on the error, the controller generates the output and the tissue containing the tumor is modelled in the block on which magnetic field is applied. After adjusting the parameters and tuning the K parameter, a step response of the system may be obtained from the scope, an example result is shown in FIG. 9.

Manual tuning methods may be used to tune the parameters of the K matrix. It can be seen from the response that the rise time of the system may be 8 seconds, and the settling time of the system may be 25 seconds. Although the obtained response is acceptable in various aspects, another issue arises regarding the presence of a steady-state error in the response. It can be seen from the graph that the response never reaches 45° C.; it reaches 44.2° C. and remains there with a steady-state error of 0.8° C. However, the error is small and may be ignored.

### Example 7: Sliding Mode Control of Magnetic Fluid Hyperthermia

The sliding mode control for hyperthermia, requires that the system be first converted into state space. After obtaining the state-space representation of the system there are two major steps in designing the control, i.e., defining the sliding surface and derivation of control input (U). The sliding surface may be defined as given below,

$$S = {( \frac{d}{dt} + λ )}^{n - 1} e$$

$$e = {T}_{d} - {T}_{m}$$

where S is the sliding surface, e is the error, T_{d} is the desired temperature value, Tₘ is the measured temperature value, and λ is the tuning parameter.

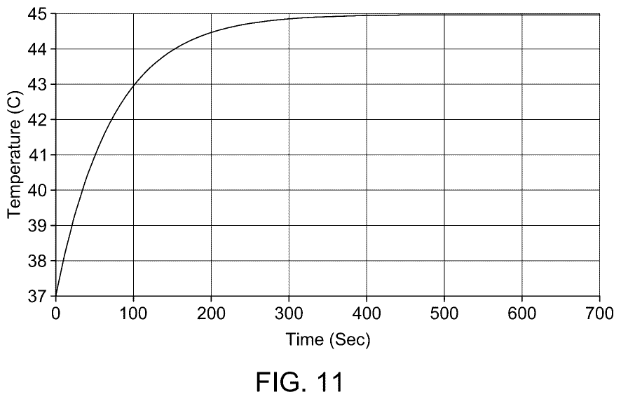

From FIG. 10, it can be seen that the error may be calculated from the pre-set temperature value and from the measured temperature value at the target site. This error may then be fed into the sliding-mode controller block. Depending on the error, the controller may then generate the output that controls magnetic ferrofluid heating. Bioheat for the tumour tissue may be modelled in this block, to which a magnetic field was applied. A step response of the system may be obtained from the scope, and one such result is shown in FIG. 11. It can be seen from the graph that there is a significant improvement in the settling time and rise time of the system.

The parameters recorded are a rise time of 170 seconds and a settling time of 380 seconds. As shown in FIG. 11, the rise time, i.e., 170 seconds is sufficient to avoid thermal shock to the tissue and the system settles down quickly after obtaining the desired value. There was no overshoot in the system, so the tissue around the tumor region was safe from the heat diffusion from the site. Similarly, the steady-state error was also eliminated, as was present in the case of the pole placement technique. The system smoothly arrives at the desired value of temperature and remains there.

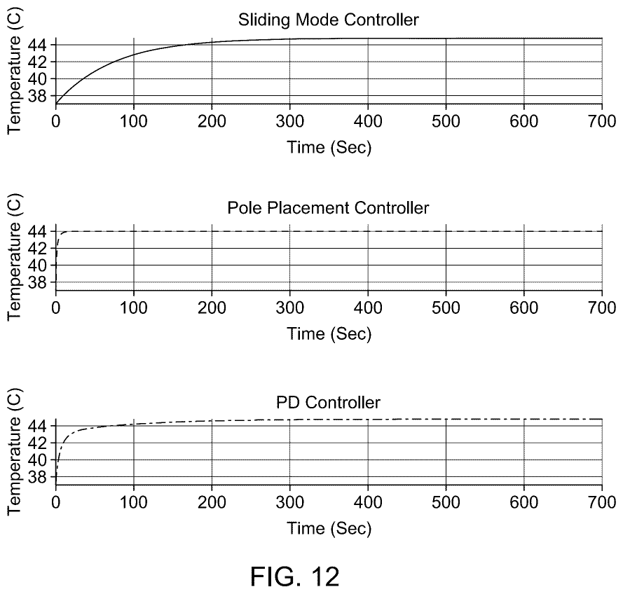

To conclude, after obtaining the response of the system via different control techniques, a comparison between them can be made. A summary of the parameters obtained from the various techniques is given in Table 2 and FIG. 12.

TABLE 2
Comparison of the control techniques

| | | Rise time | Settling time | Steady-state |
|---|---|---|---|---|
| No. | Control Technique | (s) | (s) | error (%) |
| 1 | Sliding mode control | 170 | 380 | 0 |
| 2 | Pole placement control design | 8 | 25 | 2 |
| 3 | PI controller | 100 | 500 | 0 |

PID controller of the function PI is used to achieve the results. The K_{d} gain of the PID controller was set to zero for this purpose. The sliding mode control technique provides good results with no steady-state error; however, the settling time may be large, which might result in heat diffusion to the surrounding tissues. The PID was fine-tuned to decrease the settling time; however, by reducing the settling time, the rise time also decreased, which may result in a thermal shock to tumor-affected tissues and alleged tissues. As the rise time decreased, the temperature of the tissue laden with nanoparticles rapidly increased leading to thermal shock. Moreover, the pole placement method also introduced steady-state error. Although the error may appear small, with regard to treating cancer, it is significant. The final technique used was a nonlinear control technique, i.e., sliding mode control. Sliding mode control was used to control the temperature of nanoparticles for magnetic fluid hyperthermia. This technique showed excellent results for both settling time and rise time. The steady-state error was also reduced to zero using this technique. Moreover, the rise time of the system remained within safety limits to avoid thermal shock. The settling time was also within safety limits to prevent heat diffusion to the surrounding tissues. Finally, it can be concluded that among the three techniques implemented, the sliding mode control is best suited for nanoparticle-based hyperthermia with respect to all three parameters.

Numerous modifications and variations of the present disclosure are possible in light of the above teachings. It is therefore to be understood that within the scope of the appended claims, the invention may be practiced otherwise than as specifically described herein.

1. A method for temperature control for magnetic fluid hyperthermia, comprising:
delivering therapeutic nanoparticles to a tumor site in a patient;
exciting the therapeutic nanoparticles at the tumor site with a magnetic field; and
regulating a temperature of the tumor site with a controller, wherein the controller is configured to constantly measure the temperature of the tumor site and increase or decrease the strength of the magnetic field to maintain the temperature of the tumor site within a therapeutic temperature range for a therapeutic time period;
wherein, the controller utilizes a sliding mode nonlinear control technique to regulate the temperature, the sliding mode nonlinear control technique being derived from a state space representation of a system, the system comprising the therapeutic nanoparticles, the magnetic field, and the controller.

2. The method of claim 1, wherein the temperature is regulated in a range of between 44.0 degrees Celsius (° C.) and 46.0° C.

3. The method of claim 2, wherein the therapeutic nanoparticles are excited to regulate the temperature at the tumor site to 45° C.

4. The method of claim 1, wherein the temperature that the therapeutic nanoparticles are excited to is a temperature within the therapeutic temperature range.

5. The method of claim 1, wherein the controller comprises a magnetic coil, a power source, a temperature probe, and processing circuitry.

6. The method of claim 1, wherein the rise time is less than 200 seconds, and the settling time is less than 450 seconds.

7. The method of claim 6, wherein a steady state error is zero.

8. The method of claim 1, wherein the therapeutic nanoparticles are comprised of magnetic nanoparticles.

9. The method of claim 8, wherein the magnetic nanoparticles are comprised of superparamagnetic iron oxide.

10. The method of claim 8, wherein the magnetic nanoparticles are comprised of ferrite.

11. The method of claim 1, wherein heat produced by excited therapeutic nanoparticles kills cancerous cells without killing noncancerous cells.

12. An apparatus for regulating the temperature of therapeutic nanoparticles disposed within a tumor, the apparatus comprising:
a power source configured to supply an electric current;
a magnetic coil configured to convert the electric current into a magnetic field;
a controller configured to regulate a strength of the magnetic field; and
a temperature probe configured to indirectly measure the temperature of the therapeutic nanoparticles by measuring a temperature of the tumor;
wherein,
the magnetic field may excite therapeutic nanoparticles to a target therapeutic temperature; and
the controller is further configured to maintain the temperature of the therapeutic nanoparticles at the target therapeutic temperature.

13. The apparatus of claim 12, wherein the magnetic coil is configured to excite the therapeutic nanoparticles to 45° C.

14. The apparatus of claim 12, wherein the controller utilizes a nonlinear control technique.

15. The method of claim 14, wherein the nonlinear control technique is sliding mode control.

16. The method of claim 14, wherein the sliding mode control is derived from a state space representation of a system, the system comprising the apparatus and therapeutic nanoparticles disposed within a tissue of a patient.

17. The method of claim 14, wherein a steady state error is zero.

18. A method of temperature control for magnetic fluid hyperthermia, comprising:
injecting therapeutic nanoparticles to a tumor site in a patient;
exciting the therapeutic nanoparticles with a magnetic field; and
regulating the temperature of the tumor site with a controller, the controller being configured to constantly measure the temperature of the tumor site and increase or decrease the strength of the magnetic field to maintain the temperature of the tumor site at 45° C.;
wherein,
the controller utilizes a sliding mode control derived from a state space representation of a system, the system comprising the therapeutic nanoparticles, the site of the tumor, the magnetic field, and the controller; and
the therapeutic nanoparticles are comprised of superparamagnetic iron oxide.

19. The method of claim 18, wherein a steady state error is zero.

20. The method of claim 18, wherein heat produced by excited therapeutic nanoparticles kills cancerous cells without killing noncancerous cells.
